SEQUENCE LISTING (6 sequences)

SEQ ID NO: 1 - RNA, 22 nt, Artificial Sequence
  (synthetic) let-7a
ugagguaguagguuguauaguu

SEQ ID NO: 2 - RNA, 22 nt, Artificial Sequence
  (synthetic) let-7c
ugagguaguagguuguaugguu

SEQ ID NO: 3 - DNA, 30 bp, Artificial Sequence
  (synthetic) biotin-poly(C)
cccccccccccccccccccccccccccccc

SEQ ID NO: 4 - DNA, 9 bp, Artificial Sequence
  (synthetic) Donor
actacctca

SEQ ID NO: 5 - DNA, 10 bp, Artificial Sequence
  (synthetic) Acceptor_1
aactatacaa

SEQ ID NO: 6 - DNA, 9 bp, Artificial Sequence
  (synthetic) Acceptor_2
aaccataca

(19) United States
(12) Patent Application Publication
(10) Pub. No.: US 2020/0308636 A1
(43) Pub. Date: Oct. 1, 2020

(54) Method for detecting polynucleotide using FRET-PAINT

(71) Applicant: SEOUL NATIONAL UNIVERSITY R&DB FOUNDATION, SEOUL (KR)

(72) Inventors: SUNGCHUL HOHNG, SEOUL (KR); SOOCHUL SHIN, INCHEON (KR)

(21) Appl. No.: 16/807,527

(22) Filed: Mar. 3, 2020

(30) Foreign Application Priority Data
Apr. 1, 2019 (KR) 10-2019-0037949

(51) Int. Cl. C12Q 1/6818; C12Q 1/6837; C12Q 1/6874

(52) U.S. Cl. CPC C12Q 1/6818; C12Q 1/6837; C12Q 2563/107; C12Q 2600/156; C12Q 2600/158; C12Q 1/6874

(57) ABSTRACT

The present invention relates to a method for detecting a polynucleotide at a single-molecule level using FRET-PAINT method. When using the method for detecting of the present invention, problems of non-specific binding and off-target binding can be solved, and polynucleotides that are only 1 bp mismatch can be also distinguished.

### CROSS-REFERENCE TO RELATED APPLICATIONS

This application claims the benefit of Provisional Application No. 10-2019-0037949 filed on Apr. 1, 2019 in the Korea Intellectual Property Office, the entire disclosure of which is hereby incorporated by reference.

### TECHNICAL FIELD

The present invention relates to a method for detecting a polynucleotide using FRET-PAINT method, and detecting a single nucleotide polymorphism (SNP).

### BACKGROUND ART

MicroRNAs (miRNAs) are ribonucleic acids which control protein expression and play a very important role in RNA interference, and it is very important to measure how much the certain RNA is expressed in each cell or tissue, and the like.

Conventional miRNA search techniques used a method for searching the miRNA by using a nucleotide of miRNA itself, which is fixing a miRNA to the bottom with an LNA having a complementary nucleotide thereto, and using fluorescence-labeled DNA to detect the miRNA. This method has inefficient problems because it cannot distinguish a single nucleotide polymorphism occurring on the region being fixed to the bottom, because this method uses a nucleotide of miRNA itself, and the LNA used for fixing should be different depending on a certain miRNA. In addition, because of using fluorescence-labeled DNA as a probe, there is a limitation of relatively slow detection speed.

In addition, conventional detection methods of detecting miRNAs have difficulties in detection due to small size (˜22 nt) of miRNA and high homology of family members of miRNAs with only a difference of about a single nucleotide. This is also a weak point of a standard analysis method based on PCR amplification. Accordingly, there is a need for development of miRNA detection techniques having high specificity and sensitivity.

### DISCLOSURE

### Technical Problem

In order to solve the above problems, the present invention provides a method for detecting a polynucleotide using FRET-PAINT and Total Internal Reflection Fluorescence Microscopy (TIRF).

FRET (Fluorescence Resonance Energy Transfer) refers to an energy transfer phenomenon occurring over a short distance between two fluorescent molecules. A molecule providing energy is called a donor, and a molecule receiving energy is called an acceptor. In general, they are selected as the absorption energy level of the acceptor molecule is overlapped with an emission spectrum of the donor molecule, and in this case, the energy absorbed by the donor molecule can be transferred to the acceptor molecule through dipolar interaction. To study biological molecules using FRET, the donor molecule and acceptor molecule should be attached to where needed.

FRET-PAINT (Fluorescence Resonance Energy Transfer—Point Accumulation for Imaging in Nanoscale Topography) method is a method for detecting an FRET signal when donor and acceptor oligonucleotides are combined to a target nucleic acid at the same time, by dividing the target polynucleotide into two parts and making a fluorescence-labeled oligonucleotide which is a donor to combine with one side and making a fluorescence-labeled oligonucleotide which is an acceptor to combine with the other side. Because acceptors are not excited directly and signals from excited donors are excluded by filter, FRET-PAINT can attain high SNR so that it can use at least 100 times higher imager (donor and acceptor) concentration.

Total Internal Reflection Fluorescence Microscopy (TIRF) is a device capable of observing a fluorescent material in a sub-200 nm region called Evanescence Filed, and can implement the changes in a single-molecule unit to a high resolution image. Because of obtaining an image for Evanescence Field only, not an entire image of a fluorescent sample, by using total reflection (refraction of light at the interface portion of the sample) of laser, it is possible to obtain an accurate image at the interface portion excluding interference in fluorescent light.

Up to now, single-molecule level of polynucleotide detection method or single nucleotide polymorphism detection method using the FRET-PAINT method and Total Internal Reflection Fluorescence Microscopy has not been known.

One embodiment of the present invention provides a composition of detecting a polynucleotide, comprising a nucleic acid molecule comprising 5 to 15 nucleotides having a complementary nucleic acid sequence to a target nucleic acid region of a polynucleotide to be detected, which is labeled with a donor fluorescent material, and a nucleic acid molecule comprising 5 to 15 nucleotides having a complementary nucleic acid sequence to a target nucleic acid region of a polynucleotide to be detected, which is labeled with an acceptor fluorescent material.

In addition, one embodiment of the present invention provides a method for detecting a polynucleotide comprising (1) a step of contacting the composition for detecting a polynucleotide with a biological sample comprising a polynucleotide to be detected, and (2) a step of measuring an FRET (Fluorescence Resonance Energy Transfer) signal occurring in the step of (1).

Furthermore, the present invention provides a method for detecting a single nucleotide polymorphism (SNP), comprising (1) a step of contacting the composition for detecting a polynucleotide with a biological sample comprising a polynucleotide to be detected, and (2) a step of measuring an FRET (Fluorescence Resonance Energy Transfer) signal occurring in the step (1).

### Technical Solution

Accordingly, the present inventors have found the method of detecting miRNA, which can detect the entire site of miRNA without using a complementary LNA and can measure the fluorescence signal at a single-molecule level using FRET-PAINT and total reflection, and increases the detection speed up to 10 times than conventional techniques, thereby completed the present invention.

The present invention relates to a method for detecting a specific miRNA and distinguishing a single nucleotide polymorphism (SNP), and can be applied as all techniques in various fields of bioscience such as system biology, bioinformatics, genetic engineering, and the like.

One embodiment of the present invention relates to a composition for detecting a polynucleotide, comprising a donor nucleic acid molecule which comprises a complementary nucleic acid sequence to a first target nucleic acid region of a polynucleotide to be detected and is labeled with a donor fluorescent material, and an acceptor nucleic acid molecule which comprises a complementary nucleic acid sequence to a second target nucleic acid region of the polynucleotide to be detected and is labeled with an acceptor fluorescent material, wherein the first target nucleic acid region and the second target nucleic acid region are different regions each other of the polynucleotide to be detected.

The polynucleotide to be detected is a polynucleotide targeted to be detected, and means a polynucleotide to be detected in a sample. The types of the polynucleotide to be detected may be for example, DNA, RNA or miRNA, but not limited thereto. The length of the polynucleotide to be detected may consist of or comprise 10 to 500, 10 to 400, 10 to 300, 10 to 200, 10 to 150, 10 to 100, 10 to 90, 10 to 80, 10 to 70, 10 to 60, or 10 to 50 nucleotides, but not limited thereto, and it is also possible to detect a targeted site, a polynucleotide consisting of more nucleotides can be used. The polynucleotide to be detected may be two different kinds or more, for example, 2 kinds, 3 kinds, 4 kinds, 5 kinds, 6 kinds, 7 kinds, 8 kinds, 9 kinds or 10 kinds.

The polynucleotide to be detected may have a polynucleotide tail at an end. The polynucleotide to be detected may have a modified 5′ end and/or 3′ end by poly G tailing, poly A tailing, poly U/T tailing, or poly C tailing, and the like, on purpose of binding to the surface of reaction chamber (substrate), and the like, and depending on types of the modification, biotin poly C, biotin poly U, biotin poly A, or biotin poly G, or the like may be appropriately used. When the polynucleotide to be detected is bound to the surface of reaction chamber by having a polynucleotide tail at an end, there is an advantage that loss at a target site does not occur during fixation. In addition, as all nucleotides in a sample can be fixed on one detection chip at the same time, detection can be convenient that measuring several kinds of nucleotides by changing a donor and/or nucleic acid molecule is possible.

The interval between the first target nucleic acid region and the second target nucleic acid region of the polynucleotide to be detected may be set so that the first target nucleic acid region and the second target nucleic acid region are directly adjacent, or may be set so as to have at least one of nucleotide interval. The number of nucleotides of the interval may have a lower limit of 0 or more, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or 22 or more, and a upper limit of 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, 1 or less, or 0 or less, and the interval of the first target nucleic acid region and the second target nucleic acid region may be formed in a range of the number of nucleotides to be set to a combination of the lower limit and the upper limit. For example, the interval of the first target nucleic acid region and the second target nucleic acid region may be 0 to 22, 0 to 21, 0 to 20, 0 to 19, 0 to 18, 0 to 17, 0 to 16, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 0 to 1, 0, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 1, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 2, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, or 3 nucleotides, but not limited thereto.

The composition for detecting a polynucleotide may comprise at least two different acceptor nucleic acid molecules. In this case, at least two different acceptor nucleic acid molecules may comprise a nucleic acid sequence complementary to a different target nucleic acid region of the polynucleotide to be detected. For example, when the composition comprises two different kinds of acceptor nucleic acid molecules, the donor nucleic acid molecule and the two kinds of acceptor nucleic acid molecules may cover the entire site or a targeted specific site of the polynucleotide to be detected by dividing the polynucleotide to be detected into three part, and the donor nucleic acid molecule and the two kinds of acceptor nucleic acid molecules may have an interval of at least 1 nucleotide interval, or be adjacent each other, and thereby they may be bound to the target nucleic acid region of the polynucleotide to be detected.

The at least two different kinds of acceptor nucleic acid molecules may be labeled respectively with at least two different kinds of acceptor fluorescent materials, and in this case, it can be determined that the polynucleotide to be detected has been detected when all fluorescent signals are measured.

The absorption energy level of the acceptor fluorescent materials may be overlapped with an emission spectrum of the donor fluorescent material. Specifically, the emission spectrum of the donor and the absorption spectrum of the acceptor has to be set to be overlapped, so that the acceptor can absorb the energy emitted by the donor, and as the shorter the wavelength is, the bigger the energy is, the donor and acceptor may be selected as two kinds of fluorescent materials having an overlapped energy relationship each other, in which the emission spectrum of the donor is positioned on the left (having shorter wavelength) of the absorption spectrum of the acceptor. The donor fluorescent material and the acceptor fluorescent material may be one or more selected from the group consisting of Alexa Fluor 405, Alexa Fluor 488, Cy3, Cy3.5, Cy5, Cy5.5-Allophycocyanin, Cy7, and Alexa Fluor 790, and the donor fluorescent material and the acceptor fluorescent material may be selected as different fluorescent materials each other, but not limited thereto. In the selected fluorescent materials different each other, the fluorescent material having a short emission wavelength may acts as the donor, and the fluorescent material having a long emission wavelength may act as the acceptor.

The donor nucleic acid molecule or the acceptor nucleic acid molecule may be formed to have an appropriate length, depending on a length of the polynucleotide to be detected, a length of a targeted site of the polynucleotide to be detected, a length of a target nucleic acid region of the polynucleotide to be detected, or the number of types of acceptor nucleic acid molecules comprised in the composition for detecting a polynucleotide, and the like, and for example, The donor nucleic acid molecule or the acceptor nucleic acid molecule may consist of 4 to 15, 4 to 14, 4 to 13, 4 to 12, 4 to 11, 4 to 10, 4 to 9, 4 to 8, 4 to 7, 4 to 6, 4 to 5, 5 to 15, 5 to 14, 5 to 13, 5 to 12, 5 to 11, 5 to 10, 5 to 9, 5 to 8, 5 to 7, 5 to 6, 6 to 15, 6 to 14, 6 to 13, 6 to 12, 6 to 11, 6 to 10, 6 to 9, 6 to 8, 6 to 7, 7 to 15, 7 to 14, 7 to 13, 7 to 12, 7 to 11, 7 to 10, 7 to 9, 7 to 8, 8 to 15, 8 to 14, 8 to 13, 8 to 12, 8 to 11, 8 to 10, 8 to 9, 9 to 15, 9 to 14, 9 to 13, 9 to 12, 9 to 11, 9 to 10, 10 to 15, 10 to 14, 10 to 13, 10 to 12, 10 to 11, 11 to 15, 11 to 14, 11 to 13, 11 to 12, 12 to 15, 12 to 14, 12 to 13, 13 to 15, 13 to 14, or 14 to 15 nucleotides.

The first target nucleic acid region or the second target nucleic acid region may be set so as to comprise a certain number of sequential nucleotides in the polynucleotide to be detected and may be set in an appropriate length on purpose. For example, the first target nucleic acid region or the second target nucleic acid region may consist of the same number of nucleotides as the donor nucleic acid molecule or the acceptor nucleic acid molecule.

As one example, when one kind of acceptor nucleic acid molecule is comprised in the composition for detecting a polynucleotide, the first target nucleic acid region and the second target nucleic acid region may be set so as to comprise 8 to 11 sequential nucleotides in the polynucleotide to be detected, but not limited thereto, and it may be set in an appropriate length so that the one kind of acceptor nucleic acid molecule and the donor nucleic acid molecule can cover the entire site or a targeted specific site of the polynucleotide to be detected.

As one example, when two different kinds of acceptor nucleic acid molecules are comprised in the composition for detecting a polynucleotide, the first target nucleic acid region and the second target nucleic acid region may be set so as to comprise 6 to 8 sequential nucleotides in the polynucleotide to be detected, but not limited thereto, and it may be set in an appropriate length so that the two kinds of acceptor nucleic acid molecules and the donor nucleic acid molecule can cover the entire site or a targeted specific site of the polynucleotide to be detected.

According to another embodiment of the present invention, the present invention relates to a method for detecting a polynucleotide, comprise a step of measuring a FRET (Fluorescence Resonance Energy Transfer) signal occurring by contacting the composition for detecting a polynucleotide with a biological sample.

The composition of detecting a polynucleotide may comprise a donor nucleic acid molecule which comprises a complementary nucleic acid sequence to a first target nucleic acid region of a polynucleotide to be detected and is labeled with a donor fluorescent material, and an acceptor nucleic acid molecule which comprises a complementary nucleic acid sequence to a second target nucleic acid region of the polynucleotide to be detected and is labeled with an acceptor fluorescent material, wherein the first target nucleic acid region and the second target nucleic acid region are different regions each other of the polynucleotide to be detected.

The polynucleotide to be detected may be one or more selected from the group consisting of DNA, RNA and miRNA.

The polynucleotide to be detected may be fixed on a detection chip by having a polynucleotide tail at an end.

The biological sample may comprise a polynucleotide having a similar nucleic acid sequence homology to the polynucleotide to be detected, and according to one embodiment of the present invention, when the biological sample does not comprise the polynucleotide to be detected, but comprises only a polynucleotide having a difference of one base from the polynucleotide to be detected, the biological sample may be definitely determined as not comprising the polynucleotide to be detected.

The polynucleotide having a similar nucleic acid sequence homology to the polynucleotide to be detected may have a nucleic acid sequence identity to the polynucleotide to be detected of 10% or more to less than 100%, 15% or more to less than 100%, 20% or more to less than 100%, 25% or more to less than 100%, 30% or more to less than 100%, 35% or more to less than 100%, 40% or more to less than 100%, 45% or more to less than 100%, 50% or more to less than 100%, 55% or more to less than 100%, 60% or more to less than 100%, 65% or more to less than 100%, 70% or more to less than 100%, 75% or more to less than 100%, 80% or more to less than 100%, 85% or more to less than 100%, 90% or more to less than 100%, 91% or more to less than 100%, 92% or more to less than 100%, 93% or more to less than 100%, 94% or more to less than 100%, 95% or more to less than 100%, 96% or more to less than 100%, 97% or more to less than 100%, 98% or more to less than 100%, or 99% or more to less than 100%.

Otherwise, the polynucleotide having a similar nucleic acid sequence homology to the polynucleotide to be detected may have 1 or more to 30 or less, 1 or more to 29 or less, 1 or more to 28 or less, 1 or more to 27 or less, 1 or more to 26 or less, 1 or more to 25 or less, 1 or more to 24 or less, 1 or more to 23 or less, 1 or more to 22 or less, 1 or more to 21 or less, 1 or more to 20 or less, 1 or more to 19 or less, 1 or more to 18 or less, 1 or more to 17 or less, 1 or more to 16 or less, 1 or more to 15 or less, 1 or more to 14 or less, 1 or more to 13 or less, 1 or more to 12 or less, 1 or more to 11 or less, 1 or more to 10 or less, 1 or more to 9 or less, 1 or more to 8 or less, 1 or more to 7 or less, 1 or more to 6 or less, 1 or more to 5 or less, 1 or more to 4 or less, 1 or more to 3 or less, 1 or more to 2 or less, or 1 nucleotide which is different from the polynucleotide to be detected. According to one preferable embodiment of the present invention, in case of the polynucleotide having a similar nucleic acid sequence homology to the polynucleotide to be detected having one different base from the polynucleotide to be detected, it may be distinguished from the polynucleotide to be detected accurately.

The biological sample may be an isolated cell, cell lysate, cell extract, cell fragment, isolated DNA, or isolated RNA.

The method for detecting a polynucleotide may further comprise a step of determining that the biological sample comprises the polynucleotide to be detected when a FRET signal is detected, in the step of measuring a FRET (Fluorescence Resonance Energy Transfer) signal.

It may determine having the FRET signal, when valid sm-FRET (single-molecule FRET) signal is detected in the step of measuring a FRET (Fluorescence Resonance Energy Transfer) signal. Otherwise, it may determine having the FRET signal, when valid sm-FRET (single-molecule FRET) signal is detected more than a minimum detection number, in the step of measuring a FRET (Fluorescence Resonance Energy Transfer) signal. As one example, when the sm-FRET signal over 1 second is measured twice or more, it may determine having the FRET signal. Otherwise, when an sm-FRET signal more than valid signal which has an intensity exceeding the intensity of noise is detected more than the minimum detection number, in the step of measuring a FRET signal, it may be determined to have the FRET signal. Those skilled in the art may distinguish valid sm-FRET signal having an intensity exceeding the intensity of noise. As one example, when sm-FRET signal over 1 second having an S/N (signal to noise ratio) of 3 or more is measured twice or more, it may be determined to have the FRET signal.

The valid sm-FRET signal means a signal which may be determined as valid signal having longer signal than a certain length, and may be appropriately set depending on purposes of detection.

For example, sm-FRET signal having a length of a minimum value or more may be determined as valid signal, wherein the minimum value is a minimum value of sm-FRET signal length occurring when any donor nucleic acid molecule and acceptor nucleic acid molecule which have a complementary nucleic acid sequence to a target nucleic acid region of a polynucleotide to be detected are combined with the polynucleotide to be detected. Otherwise, sm-FRET signal having longer length than a maximum value may be determined as valid signal, wherein the maximum value is a maximum value of the sm-FRET signal length occurring when any donor nucleic acid molecule and acceptor nucleic acid molecule which have a complementary nucleic acid sequence to the target nucleic acid region of the polynucleotide to be detected are combined with a polynucleotide to be excluded as a polynucleotide not to be detected. As one example, sm-FRET signal having a length of a standard value or more may be determined as valid sm-FRET signal, wherein the standard value is selected within a range set to the minimum value and the maximum value.

The valid sm-FRET signal may have a strength (intensity) exceeding the intensity of noise. The valid sm-FRET signal having a strength exceeding the intensity of noise means that sm-FRET signal having minimum or longer than signal intensity to be determined as a valid FRET signal and those skilled in the art can distinguish sm-FRET signal from noise. As one example, the intensity of the noise may be the intensity of the noise signal occurring when any donor nucleic acid molecule and acceptor nucleic acid molecule which have a nucleic acid sequence complementary to a target nucleic acid region of a polynucleotide to be detected. For example, the intensity of the noise may be maximum value of the intensities of the noise signals occurring when any donor nucleic acid molecule and acceptor nucleic acid molecule which have a nucleic acid sequence complementary to a target nucleic acid region of a polynucleotide to be detected, but those skilled in the art may distinguish an sm-FRET signal which is distinguished from noise.

The sm-FRET signal over a valid signal having an intensity exceeding the intensity of the noise may have a signal to noise ratio (S/N) of over 1, 1.1 or more, 1.2 or more, 1.3 or more, 1.4 or more, 1.5 or more, 1.6 or more, 1.7 or more, 1.8 or more, 1.9 or more, 2 or more, 2.1 or more, 2.2 or more, 2.3 or more, 2.4 or more, 2.5 or more, 2.6 or more, 2.7 or more, 2.8 or more, 2.9 or more, 3 or more, 3.1 or more, 3.2 or more, 3.3 or more, 3.4 or more, 3.5 or more, 3.6 or more, 3.7 or more, 3.8 or more, 3.9 or more, or 4 or more, and as one example, it may be 3 or more.

When the valid sm-FRET signal is detected at least the minimum number of detections, it may be determined to have a FRET signal. The minimum number of detection means the minimum number of appearance of the valid sm-FRET signal for determining that a polynucleotide to be detected is detected, and it may be appropriately set depending on detection purposes. For example, when the valid sm-FRET signal appears at least the minimum appearance number, it may determine that the polynucleotide to be detected is detected, wherein the minimum appearance number is the number of the valid sm-FRET signal occurring when any donor nucleic acid molecule and acceptor nucleic acid molecule which have a nucleic acid sequence complementary to a target nucleic acid region of a polynucleotide to be detected are combined with the target nucleic acid region of the polynucleotide to be detected. Otherwise, for a polynucleotide to be excluded as not a polynucleotide to be detected, when the valid sm-FRET signal appears at least the maximum appearance number, it may determine that the polynucleotide to be detected is detected, wherein the maximum appearance number of is the number of the valid sm-FRET signal occurring when any donor nucleic acid molecule and acceptor nucleic acid molecule which have a nucleic acid sequence complementary to a target nucleic acid region of a polynucleotide to be detected are combined with the target nucleic acid region of the polynucleotide to be detected. As one example, when the appearance number of sm-FRET signals over a valid signal is same with a stand value or more, it may determine that the polynucleotide to be detected is detected, wherein the stand value is selected within a range set to the minimum value and the maximum value. As one specific example, when an sm-FRET signal of 0.1 second or more, 0.2 seconds or more, 0.3 seconds or more, 0.4 seconds or more, 0.5 seconds or more, 0.6 seconds or more, 0.7 seconds or more, 0.8 seconds or more, 0.9 seconds or more, 1 second or more, 1.1 second or more, 1.2 seconds or more, 1.3 seconds or more, 1.4 seconds or more, 1.5 seconds or more, 1.6 seconds or more, 1.7 seconds or more, 1.8 seconds or more, 1.9 seconds or more, 2 seconds or more, 2.1 second or more, 2.2 seconds or more, 2.3 seconds or more, 2.4 seconds or more, 2.5 seconds or more, 2.6 seconds or more, 2.7 seconds or more, 2.8 seconds or more, 2.9 seconds or more, or 3 seconds or more is measured 1 times or more, 2 times or more, 3 times or more, 4 times or more, 5 times or more, 6 times or more, 7 times or more, 8 times or more, 9 times or more, or 10 times or more, it may determine that the polynucleotide to be detected is detected.

Otherwise, the valid sm-FRET signal may have a strength exceeding the intensity of noise, and for example, when sm-FRET signal having a signal to noise ratio (S/N) of over 1, 1.1 or more, 1.2 or more, 1.3 or more, 1.4 or more, 1.5 or more, 1.6 or more, 1.7 or more, 1.8 or more, 1.9 or more, 2 or more, 2.1 or more, 2.2 or more, 2.3 or more, 2.4 or more, 2.5 or more, 2.6 or more, 2.7 or more, 2.8 or more, 2.9 or more, 3 or more, 3.1 or more, 3.2 or more, 3.3 or more, 3.4 or more, 3.5 or more, 3.6 or more, 3.7 or more, 3.8 or more, 3.9 or more, or 4 or more, has a length of 1 second or more, 0.2 seconds or more, 0.3 seconds or more, 0.4 seconds or more, 0.5 seconds or more, 0.6 seconds or more, 0.7 seconds or more, 0.8 seconds or more, 0.9 seconds or more, 1 second or more, 1.1 second or more, 1.2 seconds or more, 1.3 seconds or more, 1.4 seconds or more, 1.5 seconds or more, 1.6 seconds or more, 1.7 seconds or more, 1.8 seconds or more, 1.9 seconds or more, 2 seconds or more, 2.1 second or more, 2.2 seconds or more, 2.3 seconds or more, 2.4 seconds or more, 2.5 seconds or more, 2.6 seconds or more, 2.7 seconds or more, 2.8 seconds or more, 2.9 seconds or more, or 3 seconds or more, it may be determined as the valid signal. In this case, when the valid signal having a strength exceeding the intensity of noise is measured 1 time or more, 2 times or more, 3 times or more, 4 times or more, 5 times or more, 6 times or more, 7 times or more, 8 times or more, 9 times or more, or 10 times or more, it may determine that the polynucleotide to be detected is detected.

The standard to determine that the polynucleotide to be detected is detected may be appropriately set depending on purposes of detection. For example, the standard to determine that the polynucleotide to be detected is detected may be set depending on sensitivity and specificity. As one example, in case of increasing the sensitivity, the standard value for determining as valid signal may be set close to the lower limit value, and/or, the standard value of the minimum appearance number of valid signal for determining that the polynucleotide to be detected is detected may be set close to the lower limit value. As one example, in case of increasing the specificity, the standard value for determining as valid signal may be set close to the upper limit value, and/or, the standard value of the minimum appearance number of the valid signal for determining that the polynucleotide to be detected is detected may be set close to the upper limit value.

According to other embodiment of the present invention, it relates to a method for detecting a polynucleotide, comprising a step of fixing at least two kinds or more of polynucleotides to be detected on a detection chip; a step of measuring a FRET (Fluorescence Resonance Energy Transfer) signal occurring by contacting the composition detecting one kind of the polynucleotides to be detected with the detection chip; a step of washing the detection chip; and a step of measuring a FRET (Fluorescence Resonance Energy Transfer) signal occurring by contacting the composition detecting another one kind of the polynucleotides to be detected with the detection chip. In this case, various kinds of polynucleotides which are present in a biological sample may be fixed in one chamber at the same time, and the compositions for detecting different kinds of polynucleotides are used sequentially, to detect various kinds of polynucleotides.

According to one embodiment of the present invention, when two kinds or more of polynucleotides to be detected are present in a biological sample, the compositions for detecting the corresponding polynucleotides are used sequentially, to detect polynucleotides sequentially. Specifically, various kinds of polynucleotides in a biological sample may be fixed on one detection chip, and two kinds or more of polynucleotides can be detected on the one detection chamber, not one kind of polynucleotide per one detection chamber, and because the interaction between the composition for detecting a polynucleotide according to one embodiment of the present invention and the target polynucleotide is very dynamic, polynucleotides may be sequentially detected only by replacing a probe (a nucleic acid molecule having a nucleic acid sequence complementary to a target nucleic acid region of a polynucleotide to be detected) through just flow.

Other embodiment of the present invention relates to a method for detecting a single nucleotide polymorphism (SNP), comprising a step of measuring a FRET (Fluorescence Resonance Energy Transfer) signal occurring by contacting a composition for detecting a polynucleotide with a biological sample. For example, when a FRET signal is not measured because both a donor nucleic acid molecule and an acceptor nucleic acid molecule cannot bind to the first and the second target nucleic acid regions of the polynucleotide to be detected, it may determine that a single nucleotide polymorphism or more variation is present.

### Advantageous Effects

The method for detecting a polynucleotide using FRET-PAINT of the present invention can detect the entire region of miRNA without using a complementary LNA (locked nucleic acid) to a target polynucleotide, and by using the FRET-PAINT technology, can increase the concentration of a probe up to μM units and its detection speed is up to 10 times faster than conventional techniques.

### BRIEF DESCRIPTION OF DRAWINGS

The patent or application file contains at least one drawing executed in color. Copies of this patent or patent application publication with color drawing(s) will be provided by the Patent and Trademark Office upon request and payment of the necessary fee.

FIG. 1a shows the RNA tailing method for surface fixation in the mRNA detection method according to Example 3-1.

FIG. 1b shows the design of sequences of let-7a (SEQ ID NO: 1) and let-7c (SEQ ID NO: 2), and donor (Cy3) (SEQ ID NO: 4), acceptor 1 (Cy5) (SEQ ID NO: 5), and acceptor 2 (Cy7) (SEQ ID NO: 6) strands, according to Example 3-2.

FIG. 1c shows the result of detecting Cy5 channel and Cy7 channel of let-7a and let-7c respectively on the EM-CCD camera, using FRET-PAINT comprising Total Internal Reflection Fluorescence Microscopy (TIRF), after fixing a target miRNA by hybridizing with biotin-poly (C) using poly G tailing and injecting FRET pairs (Cy3-Cy5 and Cy3-Cy7) to a detection chamber.

FIG. 1d is a drawing of schematizing the detection method of a polynucleotide using FRET-PAINT according to one embodiment of the present invention, the left is a drawing showing the case of dividing the polynucleotide to be detected into two parts and using one kind of acceptor, and the right is a drawing showing the case of dividing the polynucleotide to be detected into three parts and using two kinds of acceptors.

FIG. 2a shows the point accumulation image of Cy5 and Cy7 by FRET for 10 minutes, when 100 pM let-7a is fixed in a detection chamber and 30 nM of donor (Cy3), acceptor1 (Cy5) and acceptor2 (Cy7) strands are injected, according to Example 3-2.

FIG. 2b shows the point accumulation image by FRET for 10 minutes, when 100 pM let-7c is fixed in a detection chamber and 30 nM of donor (Cy3), acceptor1 (Cy5) and acceptor2 (Cy7) strands are injected, according to Example 3-2.

FIG. 2c shows representative intensity time traces of Cy5 (Red) and Cy7 (black) in let-7a (top) and let-7c (bottom), according to Example 3-3.

FIG. 2d shows the number of molecules showing Cy5 or Cy7 signal measured according to Example 3-3.

FIG. 3a is a drawing showing the result of detecting a polynucleotide using DNA-PAINT, and the unit of x axis is second.

FIG. 3b is a drawing showing the result of detecting the same polynucleotide as the polynucleotide detected in FIG. 3a, using FRET-PAINT, and the unit of x axis is second.

FIG. 4a is a drawing showing the result of fixing 100 pM let-7a in a detection chamber and performing substantially same as the method of Example 3-2.

FIG. 4b is a drawing which shows distinguishing let-7a and let-7c in a detection chamber in which 50 pM let-7a and 50 pM let-7c are contained together.

FIG. 4c is a drawing showing the result of fixing 100 pM let-7c in a detection chamber and performing substantially same as the method of Example 3-2.

### DETAILED DESCRIPTION OF THE EMBODIMENTS

Hereinafter, the present invention will be described in more detail through the following examples. The following examples are intended to illustrate embodiments of the present invention only, but the present invention is not limited thereto.

### Example 1. Preparation of Donor/Acceptor Strands, RNA Isolation, Poly (G) Tailing

1-1. Preparation of Donor/Acceptor Strands DNA strands, in which amine modification was introduced at the 5′ or 3′ end, were purchased from Integrated DNA technology (IDT, Coralville, Iowa). Subsequently, DNA strands were labeled with mono NHS-ester by reacting Cy5 or Cy7, with amine groups of DNA strands, respectively. The labeled DNA strands were stored in T50 buffer (10 mM Tris-HCl pH 8.0, with 50 mM NaCl). The sequences of DNAs labeled with Cy3, Cy5 or Cy7 were shown in Table 1. The DNA strand labeled with Cy3 (donor) was represented by SEQ ID NO: 4 in Table 1, and the DNA strand labeled with Cy5 (acceptor 1) was represented by SEQ ID NO: 5 in Table 1, and the DNA strand labeled with Cy7 (acceptor 2) was represented by SEQ ID NO: 6 in Table 1.

TABLE 1

| | | | SEQ |
|---|---|---|---|
| Classi- | | | ID |
| fication | Type | Sequence (5′ → 3′) | NO |
| let-7a | RNA | UGA GGU AGU AGG UUG | 1 |
| | | UAU AGU U | |
| let-7c | RNA | UGA GGU AGU AGG UUG | 2 |
| | | UAU GGU U | |
| biotin- | DNA | Biotin-CCC CCC CCC | 3 |
| poly (C) | | CCC CCC CCC CCC | |
| | | CCC CCC CCC | |
| Donor | DNA | amine-ACT ACC TCA | 4 |
| Acceptor_1 | DNA | AAC TAT ACA A-amine | 5 |
| Acceptor_2 | DNA | AAC CAT ACA-amine | 6 |

1-2. RNA Isolation and Poly (G) Tailing

miRNA was isolated using TRIzol (Invitrogen) or mirVana kit (Ambion) from HeLa cells (Korean Cell Line Bank).

After extracting and purifying an miRNA pool from a cell or tissue or serum, for Poly G tailing of 3′ end of the isolated miRNA, Poly(A) Polymerase Reaction Buffer (100 mM Tris-HCl, pH 7.0, 3.0 mM MnCl₂, 0.1 mM EDTA, 1 mM DTT, 500 μg/ml acetylated BSA, 50% glycerol) 4 μl, RNA 0.2 μM, 5 mM GTP 1 μl, 5 mM ITP 1 μl, yeast Poly(A) polymerase (Thermo Fisher) 600 units and Rnase-free water were mixed to prepare a mixture in a 20 μl volume. The isolated RNA and the mixture were cultured at 37° C. for 1 hour, and were heated at 35° C. for 15 minutes to complete the reaction.

### Example 2. Preparation of Detection Chamber Single-Molecule Experiment

2-1. Preparation of Detection Chamber

The single-molecule experiment was performed on a total internal reflection fluorescence microscope (TIRF). At first, in order to reduce inter-molecular non-specific binding, the glass surface of quartz slide was coated with the mixture of PEG and biotin-PEG at a ratio of 40:1, and double-sided adhesive tape was attached between the quartz slide and glass coverslip, to prepare a detection chamber.

2-2. Single-Molecule Experiment

After culturing miRNAs having Poly (G) tailing prepared in Example 1-2 with biotinylated poly (C) DNA strands (30 nt), they were fixed in the detection chamber prepared in Example 2-1 through streptavidin-biotin interaction.

Then, donor (Cy3) strand 30 mM, acceptor 1 (Cy5) strand 20 nM, and acceptor 2 (Cy7) strand 30 nM prepared in Example 1-1 which was contained in Imaginf buffer (20 mM Tris-HCl (pH 8.0) with 135 mM KCl, 0.5% formamide, 120 mM UREA, 100 mM NaCl, and oxygen scavenger system: 4 mg/ml D-(+)-glucose (Sigma-Aldrich), 1 mg/ml glucose oxidase (Sigma-Aldrich), 0.04 mg/ml catalase (Roche), and saturated Trolox (25 mg/50 mL)) were injected in the detection chamber. The experiment was performed at 30° C. Cy3 was excited with 532-nm laser (Compass215M, Coherent, Santa Clara, Calif.), and the fluorescence signals of Cy5 and Cy7 were collected through a water-immersion object lens (UPlanSApo 60X, Olympus), and were imaged with an EM-CCD camera (Ixon DV897, Andor). The data were collected using a home-built program written in Visual C++ (Microsoft), and were analyzed using MATLAB (R2010a, The MathWorks) and Origin (8.0, OriginLab).

### Example 3. miRNA Detection Using FRET-PAINT Method

3-1. miRNA Tailing for Surface Fixation

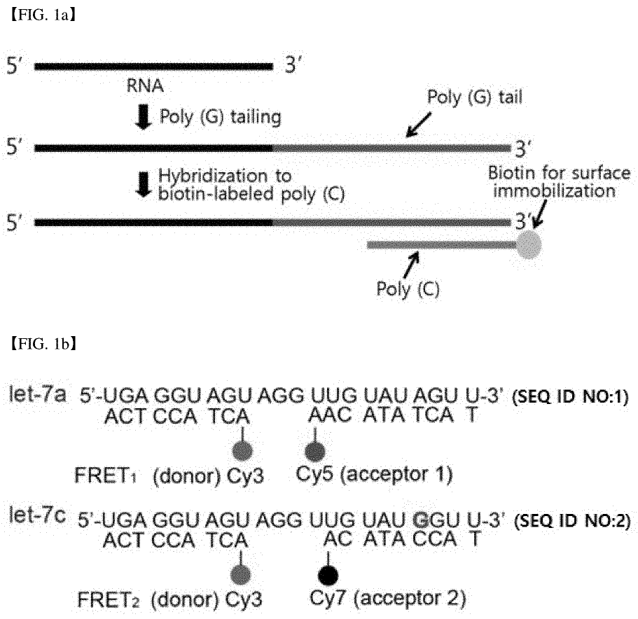

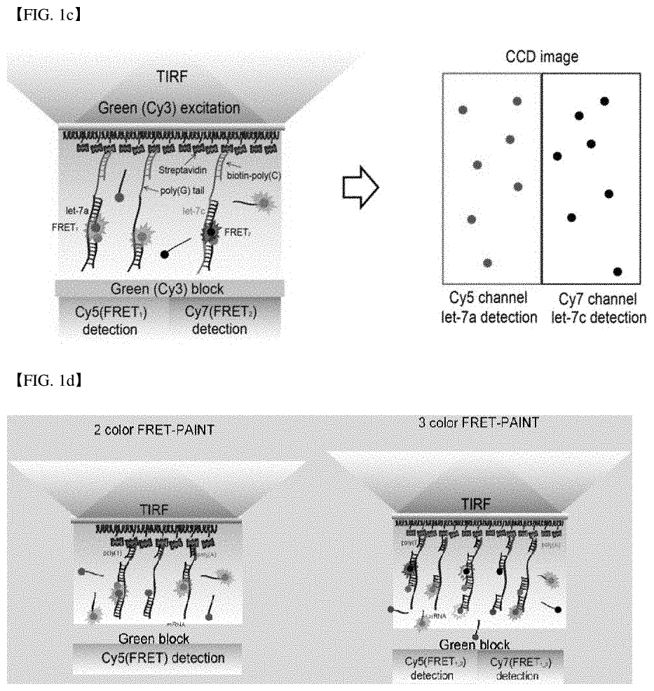

As the first step, a step of adding the poly(G) tail to RNA extracted by the method of Example 1-2 was performed (FIG. 1a). The poly(G) tail added to RNA was used for fixing RNAs extracted by the method of the present invention on the surface of the detection chamber at the same time. Then, in order to capture miRNA in the detection chamber through streptavidin-biotin interaction, biotinylated poly(C) DNA strands were fused with the poly(G) RNA tail. This series of processes were shown in FIG. 1a.

3-2. Validity Test of miRNA Detection Method

In order to test the validity of the miRNA detection method, at first, let-7a and let-7c which were let-7 family were used. In let-7a and let-7c, only a single nucleotide is different (FIG. 1b). The sequences of let-7a and let-7c were represented by SEQ ID NO: 1 and SEQ ID NO: 2, respectively.

Then, the donor (Cy3) strand (SEQ ID NO: 3) complementary to 5′ region of let-7a (SEQ ID NO: 1) and let-7c (SEQ ID NO: 2) was prepared, and the acceptor 1 (Cy5) strand (SEQ ID NO: 4) complementary to 3′ region of let-7a and the acceptor 2 (Cy7) strand (SEQ ID NO: 5) complementary to 3′ region of let-7c were prepared (See FIG. 1b). The donor and acceptors were designed so that the donor and acceptors exhibit a high FRET state when they bind to a target instantly and bind to the target at the same time.

Subsequently, using FRET-PAINT comprising total internal reflection fluorescence microscopy (TIRF), let-7a and let7c were detected in the Cy5 channel and Cy7 channel on an EM-CCM camera, respectively, and the result was shown in FIG. 1c. Specifically, after fixing target miRNAs by hybridizing with biotin-poly C using poly G tailing according to the method of Example 3-1, FRET pairs (Cy3-Cy5 and Cy3-Cy5) were injected into a detection chamber. The Cy3-Cy5 pair used let-7a for detection, and the Cy3-Cy7 pair used let-7c for detection. Cy3 was excited by a green laser, and Cy5 and Cy7 signals by FRET were imaged by the EM-CCD camera, respectively. The Cy3 signal was rejected and the background noise was significantly reduced.

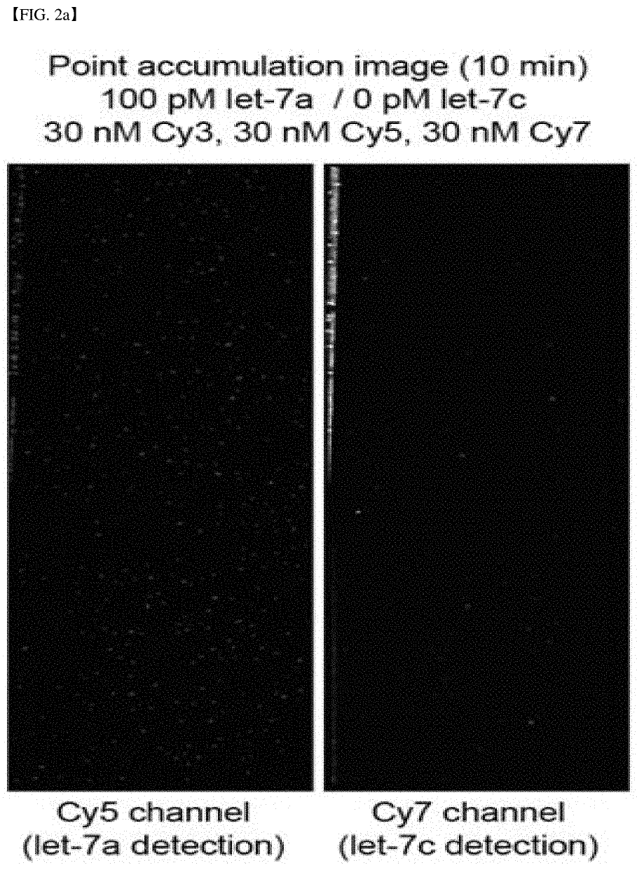

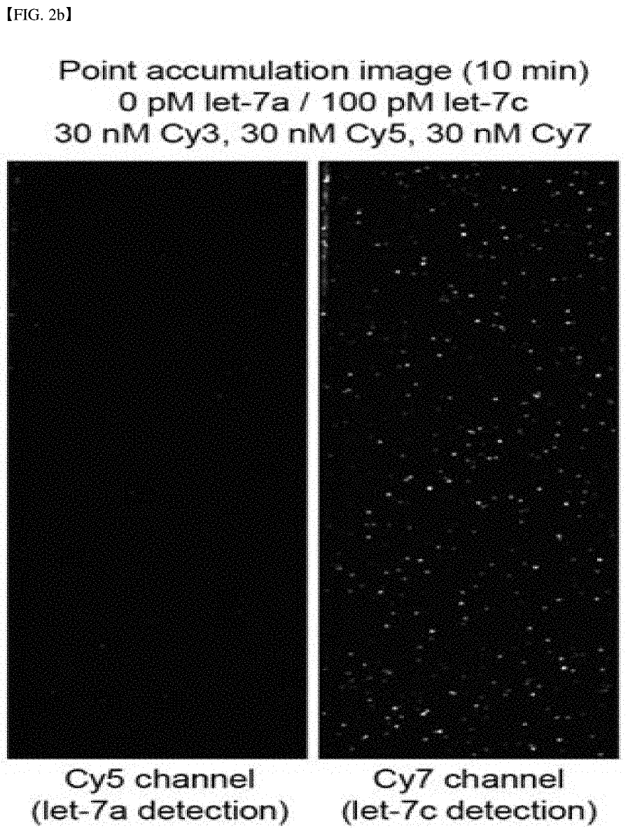

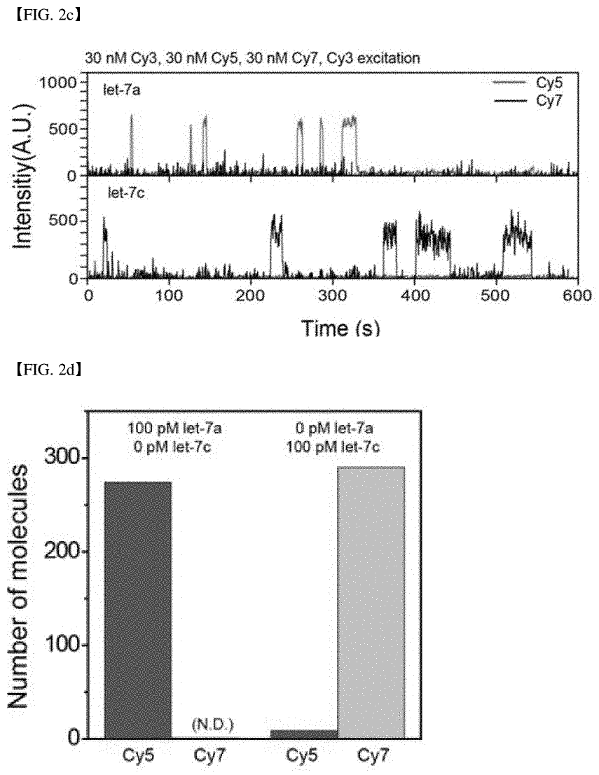

Then, 100 pM of let-7a with poly (G) tailing was fixed in the detection chamber, and the donor (Cy3) strand 30 nM, acceptor 1 (Cy5) strand 30 nM, and acceptor 2 (Cy7) strand 30 nM were injected into the detection chamber. After that, the point accumulation image of Cy5 and Cy7 by FRET for 10 minutes was shown in FIG. 2a. As shown in FIG. 2a, in the FRET-PAINT method, the fluorescent signal from the donor was blocked, and only the acceptor signal passing through FRET was measured, and the significantly low background noise occurred. When let-7a was fixed, only the Cy5 signal was observed.

In contrast, when 100 pM of let-7c was fixed, only the Cy7 signal was observed, and this was shown in FIG. 2b. FIG. 2b shows the point accumulation image by FRET for 10 minutes, after fixing let-7c of 100 pM and injecting the donor (Cy3), acceptor 1 (Cy5) and acceptor 2 (Cy7) strands of 30 nM.

From the result, it was confirmed that the detection method of the present invention could distinguish let-7c and let-7a successfully.

3-3. Measurement of Representative Fluorescence Intensity Time Traces and Quantitative Analysis of Number of Molecules

Representative fluorescence intensity time traces of Cy5 and Cy7 were measured. The intensity of fluorescence recorded on the CCD camera was measured over time, and the result was shown in FIG. 2c.

FIG. 2c shows representative fluorescence intensity time traces of Cy5 and Cy7. The Cy5 and Cy7 signals were observed in let-7a and let-7c, respectively, and each of them were shown exclusively.

Then, quantitative analysis was conducted by counting the number of molecules showing the Cy5 signal or showing the Cy7 signal among fluorescence intensity time traces of each molecule, and the result was shown in FIG. 2d.

As shown in FIG. 2d, 274 molecules showed the Cy5 signal in 100 pM let-7a, and 290 molecules showed the Cy7 signal in 100 pM let-7c. On this wise, the calibration capable of predicting the concentration when detecting let-7a at an unknown concentration through quantitative analysis could be established.

### Example 4: Polynucleotide Detection Using DNA-PAINT

In order to confirm the fast detection speed of polynucleotide detection using FRET-PAINT, the detection speed was compared with DNA-PAINT.

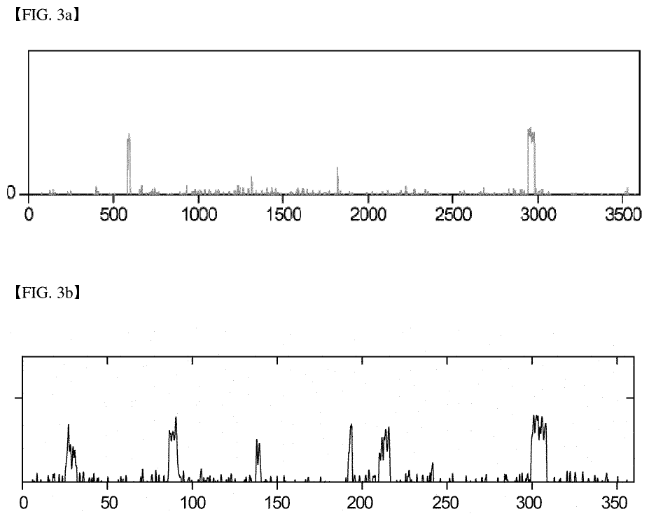

Specifically, in order to detect a polynucleotide using DNA-PAINT, a complementary DNA probe to a target nucleic acid region was designed, and an experiment was conducted so that fluorescence was recorded on a CCD camera when the polynucleotide and DNA probe were combined. The result was shown in FIG. 3a.

In addition, by the substantially same method as Example 3-3, the same polynucleotide was detected using FRET-PAINT, and the result was shown in FIG. 3b.

As a result, it could be confirmed that the detection method of a polynucleotide using FRET-PAINT according to the present invention showed at least 10 times faster detection speed than the method using DNA-PAINT, as the detection time took 3600 seconds or more of detection time in case of DNA-PAINT, but the detection time took 360 seconds or less in case of FRET-PAINT.

### Example 5. SNP Detection Using FRET-PAINT

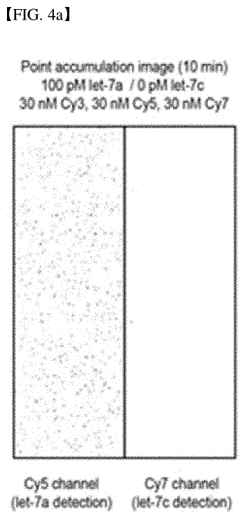

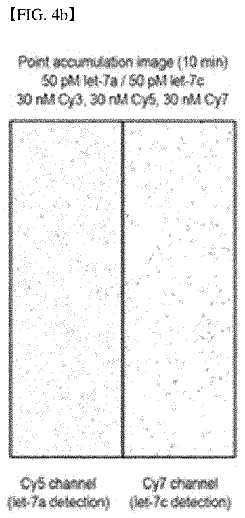

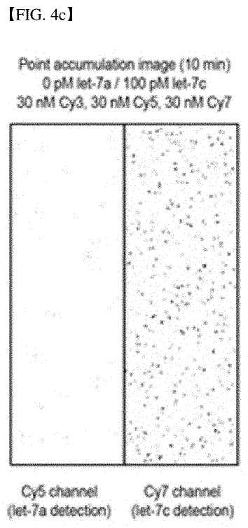

In order to confirm whether it is possible to detect a single nucleotide polymorphism (SNP) using FRET-PAINT, in addition to the case of fixing 100 pM let-7c or 100 pM let-7a was fixed in a detection chamber, the substantially same method as Example 3-2 was performed when 50 pM let-7a and 50 pM let-7c were contained together in a detection chamber to confirm whether let-7a and let-7c could be distinguished. The result was shown in FIG. 4a to FIG. 4c.

As a result, it could be confirmed that only let-7a and let-7c were successfully detected in FIG. 4a and FIG. 4c respectively, and in addition, it was confirmed that a single nucleotide polymorphism (SNP) could be detected using FRET-PAINT, as let-7a and let-7c in which only one nucleotide was different could be distinguished even when let-7a and let-7c were present together (FIG. 4b).

1. A composition for detecting a polynucleotide, comprising
a donor nucleic acid molecule which comprises a complementary nucleic acid sequence to a first target nucleic acid region of a polynucleotide to be detected and is labeled with a donor fluorescent material, and
an acceptor nucleic acid molecule which comprises a complementary nucleic acid sequence to a second target nucleic acid region of the polynucleotide to be detected and is labeled with an acceptor fluorescent material,
wherein the first target nucleic acid region and the second target nucleic acid region are different regions of the polynucleotide to be detected.

2. The composition according to claim 1, wherein the interval of the first target nucleic acid region and the second target nucleic acid region is 0 to 22 nucleotides.

3. The composition according to claim 1, comprising two or more different kinds of acceptor nucleic acid molecules.

4. The composition according to claim 3, wherein the two or more different kinds of acceptor nucleic acid molecules are labeled with different acceptor fluorescent materials to each other.

5. The composition according to claim 1, wherein the absorption energy level of the acceptor fluorescent material is overlapped with an emission spectrum of the donor fluorescent material.

6. The composition according to claim 1, wherein the donor fluorescent material and the acceptor fluorescent material are one or more kinds selected from the group consisting of Alexa Fluor 405, Alexa Fluor 488, Cy3, Cy3.5, Cy5, Cy5.5-Allophycocyanin, Cy7, and Alexa Fluor 790, and
the absorption energy level of the acceptor fluorescent material is overlapped with an emission spectrum of the donor fluorescent material.

7. The composition according to claim 1, wherein the polynucleotide to be detected is fixed on a detection chip, by having a polynucleotide tail at the end.

8. The composition according to claim 1, wherein the polynucleotide to be detected is one or more kinds selected from the group consisting of DNA, RNA, and miRNA.

9. The composition according to claim 1, wherein each of the donor nucleic acid molecule and the acceptor nucleic acid molecule consists of 5 to 15 nucleotides.

10. The composition according to claim 1, wherein each of the first target nucleic acid region and the second target nucleic acid region comprises sequential 5 to 15 nucleotides in the polynucleotide to be detected.

11. The composition according to claim 3, wherein two different kinds of the acceptor nucleic acid molecules are comprised, and each of the first target nucleic acid region and the second target nucleic acid region comprise 6 to 8 sequential nucleotides in the polynucleotide to be detected.

12. A method for detecting a polynucleotide, comprising a step of measuring a FRET (Fluorescence Resonance Energy Transfer) signal occurring by contacting the composition for detecting a polynucleotide of claim 1 with a biological sample.

13. The method according to claim 12, wherein the polynucleotide to be detected is one or more kinds selected from the group consisting of DNA, RNA, and miRNA.

14. The method according to claim 12, wherein the polynucleotide to be detected is fixed on a detection chip by having a polynucleotide tail at the end.

15. The method according to claim 12, wherein the biological sample comprises a polynucleotide having at least 90% base identity to the polynucleotide to be detected.

16. The method according to claim 12, wherein the biological sample is an isolated cell, cell lysate, cell extract, cell fragment, isolated DNA or isolated RNA, which comprise the polynucleotide to be detected.

17. The method according to claim 12, further comprising a step of determining the biological sample comprises the polynucleotide to be detected when valid sm-FRET (single molecule-Fluorescence Resonance Energy Transfer) signal is detected,
wherein the valid sm-FRET signal is a signal longer than maximum value of FRET signal length occurring when any donor nucleic acid molecule and acceptor nucleic acid molecule which have a complementary nucleic acid sequence to the target nucleic acid region of the polynucleotide to be detected are combined with any polynucleotide which is not the polynucleotide to be detected.

18. The method according to claim 12, further comprising a step of determining the biological sample comprises the polynucleotide to be detected when valid sm-FRET (single molecule-Fluorescence Resonance Energy Transfer) signal is detected more than a minimum detection number,
wherein the valid sm-FRET signal is a signal longer than maximum value of FRET signal length occurring when any donor nucleic acid molecule and acceptor nucleic acid molecule which have a complementary nucleic acid sequence to the target nucleic acid region of the polynucleotide to be detected are combined with any polynucleotide which is not the polynucleotide to be detected, and
wherein the minimum detection number is a maximum appearance number of the valid sm-FRET signal occurring when any donor nucleic acid molecule and acceptor nucleic acid molecule which have a complementary nucleic acid sequence to a target nucleic acid region of the polynucleotide to be detected are combined with any polynucleotide which is not the polynucleotide to be detected.

19. A method for detecting a polynucleotide, comprising
a step of fixing at least two kinds of polynucleotides to be detected on a detection chip;
a step of measuring a FRET (Fluorescence Resonance Energy Transfer) signal occurring by contacting the composition of claim 1 which is detecting one of the at least two kinds of polynucleotides to be detected with the detection chip;
a step of washing the detection chip; and
a step of measuring a FRET (Fluorescence Resonance Energy Transfer) signal occurring by contacting the composition of claim 1 which is detecting another one kind of the at least two kinds of polynucleotides to be detected with the detection chip.

20. A method for detecting a single nucleotide polymorphism (SNP), comprising a step of measuring a FRET (Fluorescence Resonance Energy Transfer) signal occurring by contacting the composition for detecting a polynucleotide of claim 1 with a biological sample.
